(19) United States
(12) Patent Application Publication
(10) Pub. No.: US 2017/0128381 A1
(43) Pub. Date: May 11, 2017

(54) STIMULUS-RESPONSIVE CORE-SHELL PARTICLES

(71) Applicant: AGENCY FOR SCIENCE, TECHNOLOGY AND RESEARCH, Singapore (SG)

(72) Inventors: Jatin Nitin KUMAR, Singapore (SG); Connie Kin Man LIU, Singapore (SG); Ye LIU, Singapore (SG); Xian Jun LOH, Singapore (SG)

(21) Appl. No.: 15/320,745

(22) PCT Filed: Jun. 22, 2015
(86) PCT No.: PCT/SG2015/050174

§ 371 Date: Dec. 20, 2016

(30) Foreign Application Priority Data
Jun. 20, 2014 (SG) 10201403499U

(51) Int. Cl. A61K 9/51; C08F 292/00

(52) U.S. Cl. CPC A61K 9/5138; A61K 9/5192; C08F 292/00

(57) ABSTRACT

There is provided a core-shell particle having pores extending through its shell and a plurality of polymers that are bonded to the outer surface of the shell, wherein the polymers are comprised of repeating monomer units of formula (1): [Formula should be inserted here] wherein the substituents are as defined herein. There is also provided a method of synthesizing the core-shell particle and use of the core-shell particle as a delivery agent.

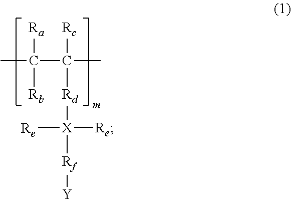

### TECHNICAL FIELD

The present invention generally relates to a core-shell particle that is able to respond to an external stimulus. The present invention also relates to a method for synthesizing the core-shell particle and to uses thereof.

### BACKGROUND ART

Encapsulation systems are needed in many areas such as consumer care and pharmaceuticals where these encapsulation systems are needed for protection, controlled release and delivery of various compounds. A suitable encapsulation system should provide secure encapsulation of targeted compounds but also be able to release the encapsulated compounds when needed. To meet this requirement, it is necessary to develop stimuli-responsive encapsulation systems which are stable without the presence of stimuli but are disassembled to release the encapsulated compounds when exposed to the stimuli.

Of particular interest are systems which securely encapsulate active species at low temperatures, and release the active species at higher temperatures. Thermally triggered systems have many implications ranging from drug delivery to consumer care whereby the release of active compounds is desired at physiological temperatures.

Another form of stimuli-responsive encapsulation system that would be of interest would be one that responds to the presence of carbon dioxide. During the event of cardiac arrest or other circumstances where a patient stops breathing, doctors are allowed a very brief timeframe in which to take action before there is damage to the patient's brain, eventually leading to death. This is caused by rapidly increasing carbon dioxide and diminishing oxygen supply in the blood to the brain. If doctors are unable to resuscitate the patient, the therapeutic options are almost non-existent. Therefore, it is presently a challenge, especially in emergency cases outside of a hospital setting, to minimize mortality from such situations. This phenomenon would also extend to other forms of ischemia where blood supply to vital tissue may be blocked. Machines that artificially oxygenate of blood and remove carbon dioxide are the only viable strategy; however they are marred by logistical limitations and are very costly. Other oxygen encapsulation systems can be oxygen filled lipid microspheres which only deliver oxygen, but are not responsive to carbon dioxide at all. In addition, such oxygen filled lipid microspheres have to be prepared fresh and cannot be stored for long periods of time. Hence, there is an unmet need for a stimulus-responsive encapsulation system that overcomes, or at least ameliorates, one or more of the disadvantages described above.

### SUMMARY OF INVENTION

According to a first aspect, there is provided a core-shell particle with pores extending through its shell and a plurality of polymers that are bonded to the outer surface of the shell, wherein said polymers are comprised of repeating monomer units of formula (1):

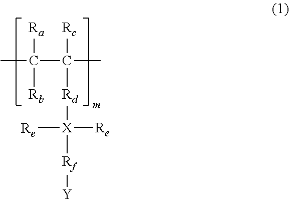

wherein
m is at least 1;
Rₐ, R_{b}, R_{c}, and Rₑ are independently selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl;
R_{d} is selected from the group consisting of a bond, optionally substituted hetero-aliphatic, optionally substituted aliphatic group, optionally substituted alkoxy, optionally substituted ester, carbonyl, optionally substituted amide, optionally substituted aryl, optionally substituted heteroaryl, optionally substituted cycloalkyl and optionally substituted heterocycloalkyl;
X is selected from the group consisting of a heteroatom or a positively charged ion;
R_{f} and Y are absent when X is a heteroatom but are both present when X is a positively charged ion such that R_{f} is selected from the group consisting of optionally substituted aliphatic, optionally substituted hetero-aliphatic, optionally substituted aryl, optionally substituted heteroaryl, optionally substituted cycloalkyl and optionally substituted heterocycloalkyl; and Y is a negatively charged ion when bonded to R_{f}.

The polymers grafted on the core-shell particle may respond to an external stimulus. When exposed to an external stimulus, the polymer brushes on the core-shell particle may uncoil (that is, extend) or may collapse to form an impermeable layer on the shell. When the polymer brushes uncoil, the pores of the particle may be exposed to the external environment, thus allowing the exchange of the core contents between the core of the particle and the external environment and vice versa. When the polymer brushes collapse to form a layer on the shell, the pores of the particle may be blocked or sealed by the polymeric layer such that there is no exchange of the core contents between the core of the particle and the external environment and vice versa. Hence, the core-shell particle may act as a delivery agent that can release the encapsulated contents in the core as required in an appropriate environment.

According to a second aspect, there is provided a method of synthesizing a core-shell particle as defined herein, comprising the steps of:
i) providing a halide-functionalized particle; and
ii) reacting said halide-functionalized particle with a monomer of Formula (1) to form said core-shell particle.

According to a third aspect, there is provided a method of transferring an agent into or out of the core of the core-shell particle as defined herein, comprising the step of altering the temperature or carbon dioxide concentration of a solution that is in contact with said core-shell particle.

According to a fourth aspect, there is provided a method of loading a gas into the core of the core-shell particle as defined herein, comprising the step of exposing the core-shell particle to a gaseous environment containing the gas and allowing the gas to permeate into the core of the core-shell particle.

According to a fifth aspect, there is provided a delivery agent comprising a plurality of core-shell particles as defined herein, wherein the core of said particles contains an agent.

According to a sixth aspect, there is provided a pharmaceutical composition comprising a plurality of core-shell particles as defined herein, wherein the core of said particles contains an agent.

### DEFINITIONS

The following words and terms used herein shall have the meaning indicated:

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

The term “aliphatic” is one that includes only carbon and hydrogen and possibly having monovalent and divalent straight or branched chain unsaturated hydrocarbon groups from 1 to 50 carbon atoms, 1 to 30 carbon atoms, 1 to 12 carbon atoms, 1 to 10 carbon atoms, or 1 to 6 carbon atoms, or any of the number of carbon atoms falling within the stated range. In cases where there is at least one double bond, either the E, Z, cis or trans stereochemistry may exist, where applicable, at any location along the carbon chain. An aliphatic group as used herein may be a terminal or bridging group.

The term “alicyclic” refers to a group that is both aliphatic and cyclic having from 3 to 30 carbon atoms or any of the number of carbon atoms falling within the stated range. They contain one or more all-carbon rings which may be either saturated or unsaturated, but do not have aromatic character. Examples may include monocyclic cycloalkanes such as cyclopropane, cyclobutane, cyclopentane, cyclohexane, cycloheptane, cyclooctane, and so on. Bicyclic alkanes may include bicycloundecane and decalin. Polycyclic alkanes may include cubane, basketane, and housane. Such a group as used herein may be a terminal or bridging group.

The term “aromatic” as used herein refers to monovalent and divalent single, polynuclear, conjugated and fused residues of aromatic hydrocarbons having from 6 to 30 carbon atoms, or any of the number of carbon atoms falling within the stated range. Examples of such groups include phenyl, biphenyl, naphthyl, phenanthrenyl, and the like. An aromatic group as used herein may be a terminal or bridging group.

The term “arylaliphatic” means an aryl-aliphatic-group in which the aryl and aliphatic moieties are as defined herein. Preferred arylalkyl groups contain a C₁₋₃₀ alkyl moiety. Exemplary arylalkyl groups include benzyl, phenethyl, 1-naphthalenemethyl and 2-naphthalenemethyl. The group as used herein may refer to a terminal or bridging group.

The term “halogen” represents a chlorine, fluorine, bromine or iodine atom. Likewise, the term “halide” or “halo” represents a chloride, fluoride, bromide or iodide.

The term “heteratom” as used herein may refer to any suitable atom selected from N, O, S, Se and Si.

The term “alkylene” as used herein represents a substituent group or part of a group that refers to a straight or branched saturated aliphatic hydrocarbon group, such as C₁₋₃₀alkylene, C₁₋₁₂alkylene, C₁₋₁₀alkylene, or C₁₋₆alkylene or any of the number of carbon atoms falling within the stated range. Where the “alkylene” is a terminal group, this term may be used interchangeably with the term “alkyl”. Examples of suitable straight and branched C₁₋₆alkylene substituents include methyl, ethyl, n-propyl, 2-propyl, n-butyl, sec-butyl, t-butyl, hexyl, and the like. The group may be a terminal group or a bridging group.

The term “heteroalkylene” as used herein means a hetero-alkylene-group which contains one or more heteroatoms with the alkylene moiety as defined herein.

The term “acyl” includes within its meaning an R—C(═O)— group in which the R group may be an alkyl, cycloalkyl, heterocycloalkyl, aryl or heteroaryl group as defined herein. Examples of acyl include acetyl and benzoyl. The group may be a terminal group or a bridging group. If the group is a terminal group it is bonded to the remainder of the molecule through the carbonyl carbon.

The term “ester” includes within its meaning an R—COO— group in which the R group may be a hydrogen, alkyl, cycloalkyl, heterocycloalkyl, aryl or heteroaryl group as defined herein. The group may be a terminal group or a bridging group. If the group is a terminal group it is bonded to the remainder of the molecule through the oxygen forming an ester linkage.

The term “amide” includes within its meaning an R—CONR—R group in which the R group may be a hydrogen, alkyl, cycloalkyl, heterocycloalkyl, aryl or heteroaryl group as defined herein. The group may be a terminal group or a bridging group. If the group is a terminal group it is bonded to the remainder of the molecule through the carbon or nitrogen forming an amide linkage.

The term “carbonyl” includes within its meaning an R—C(═O)R group in which the R group may be a hydrogen, alkyl, cycloalkyl, heterocycloalkyl, aryl or heteroaryl group as defined herein. The group may be a terminal group or a bridging group. If the group is a terminal group it is bonded to the remainder of the molecule through the carbon.

“Alkoxy” or “Alkyloxy” refers to an alkyl-O— group in which alkyl is as defined herein. These terms may be used interchangeably. Preferably the alkyloxy is a C₁-C₁₀alkyloxy. Examples include, but are not limited to, methoxy and ethoxy. The group as used herein refers to a terminal group.

“Alkenylene” as a group or part of a group denotes an aliphatic hydrocarbon group containing at least one carbon-carbon double bond and which may be straight or branched such as C₂₋₃₀alkenylene, C₂₋₁₂alkenylene, C₂₋₁₀alkenylene, or C₂₋₆alkenylene, or having any of the number of carbon atoms falling within the stated range in the normal chain. The group may contain a plurality of double bonds in the normal chain and the orientation about each is independently E, Z, cis or trans where applicable. Where the “alkenylene” is a terminal group, this term may be used interchangeably with the term “alkenyl”. Exemplary alkenylene groups include, but are not limited to, ethenyl, propenyl, butenyl, pentenyl, hexenyl, heptenyl, octenyl and nonenyl.

It is understood that alkenylene groups may be isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

The term “heteroalkenylene” as used herein means a hetero-alkenylene-group which contains one or more heteroatoms with the alkenylene moiety as defined herein.

“Alkynylene” as a group or part of a group means an aliphatic hydrocarbon group containing a carbon-carbon triple bond and which may be straight or branched such as C₂₋₃₀alkynylene, C₂₋₁₂alkynylene, C₂₋₁₀alkynylene, or C₂₋₆alkynylene, or having any of the number of carbon atoms falling within the stated range in the normal chain. Where the “alkynylene” is a terminal group, this term may be used interchangeably with the term “alkynyl”. Exemplary structures include, but are not limited to, ethynyl and propynyl.

The term “heteroalkynylene” as used herein means a hetero-alkynylene-group which contains one or more heteroatoms with the alkynylene moiety as defined herein.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₁₂ alkyl group. The group as used herein refers to a terminal group.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group as used herein refers to a terminal group. Typically an aryl group is a C₆-C₁₈ aryl group.

“Heterocycloalkyl” refers to a saturated monocyclic, bicyclic, or polycyclic ring containing at least one heteroatom selected from N, O, S, Se and Si, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₂-C₁₂ heterocycloalkyl group. The group as used herein refers to a terminal group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include N, O, S, Se and Si. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. A heteroaryl group is typically a C₁-C₁₈ heteroaryl group. The group as used herein refers to a terminal group.

A “bond” is a linkage between atoms in a compound or molecule. The bond may be a single bond, a double bond, or a triple bond.

The term “optionally substituted” as used herein means the group to which this term refers may be unsubstituted, or may be substituted with one or more groups independently selected from halogen, heteroatom, alkyl, acyl, ester, carbonyl, alkyloxy, alkenyl, alkynyl, sulfonamide, aminosulfonamide, cycloalkyl, aryl, heterocycloalkyl and an heteroaryl.

The term “polymer” or “polymeric” as used herein refers to a molecule having two or more monomeric repeat units. It includes linear and branched polymer structures, and also encompasses cross-linked polymers as well as copolymers (which may or may not be cross-linked), thus including block copolymers, alternating copolymers, random copolymers, graft copolymers and the like.

The term “silica” as used herein refers to oxide of silicon having the approximate chemical formula SiO₂, without regard to shape, morphology, porosity, and water or hydroxyl content.

The term “nano” as used herein, when referring to a dimension or a parameter, refers to the size of that dimension or parameter being in the nano-range, or less than about 1000 nm, less than about 500 nm, less than about 200 nm or less than about 100 nm.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF OPTIONAL EMBODIMENTS

Exemplary, non-limiting embodiments of a core-shell particle will now be disclosed. The core-shell particle has pores that extend through its shell and comprises a plurality of polymers that are bonded to the outer surface of the shell, wherein the polymers are comprised of repeating monomer units of formula (1):

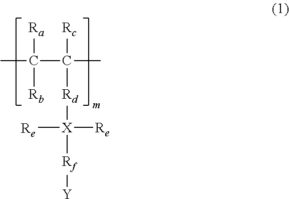

wherein
m is at least 1;
Rₐ, R_{b}, R_{c}, and Rₑ are independently selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl;
R_{d} is selected from the group consisting of a bond, optionally substituted hetero-aliphatic, optionally substituted aliphatic group, optionally substituted alkoxy, optionally substituted ester, carbonyl, optionally substituted amide, optionally substituted aryl, optionally substituted heteroaryl, optionally substituted cycloalkyl and optionally substituted heterocycloalkyl;
X is selected from the group consisting of a heteroatom or a positively charged ion;
R_{f} and Y are absent when X is a heteroatom but are both present when X is a positively charged ion such that R_{f} is selected from the group consisting of optionally substituted aliphatic, optionally substituted hetero-aliphatic, optionally substituted aryl, optionally substituted heteroaryl, optionally substituted cycloalkyl and optionally substituted heterocycloalkyl; and Y is a negatively charged ion when bonded to R_{f}.

The polymer may be bonded to the outer surface of the shell at either end of the polymer.

The optionally substituted aliphatic may be selected from the group consisting of optionally substituted C₁₋₃₀alkylene, optionally substituted C₂₋₃₀alkenylene and optionally substituted C₂₋₃₀alkynylene.

The optionally substituted hetero-aliphatic may contain one or more heteroatoms selected from N, O, S, Se or Si, and may be selected from the group consisting of optionally substituted C₁₋₃₀-heteroalkylene, optionally substituted C₂₋₃₀-heteroalkenylene and optionally substituted C₂₋₃₀-heteroalkynylene.

In the core-shell particle, X may be selected from the group consisting of N, N⁺ and P³⁺. When X is N⁺ or P³⁺, R_{f} and Y may be present and Y may be selected from the group consisting of sulfonate, carboxylate, nitrite and carbonite. R_{f} may be selected from the group consisting of C₁-alkylene, C₂-alkylene, C₃-alkylene, C₄-alkylene, C₅-alkylene and C₆-alkylene.

Rₐ, R_{b}, R_{c}, and Rₑ may independently be a hydrogen or an optionally substituted aliphatic. In an embodiment, all of Rₐ, R_{b} and Rₑ are hydrogen. Rₐ, R_{b}, R_{c} and Rₑ may be an optionally substituted aliphatic such as optionally substituted C₁₋₃₀alkyl, optionally substituted C₁₋₁₂alkyl, optionally substituted C₁₋₁₀alkyl, or optionally substituted C₁₋₆alkyl. Rₐ, R_{b}, R_{c} and Rₑ may independently be methyl, ethyl, propyl, butyl, pentyl, hexyl, heptyl, octyl, nonyl decyl, undecyl or dodecyl. Each of the Rₐ, R_{b}, R_{c} and Rₑ may be the same or may be different from each other. Where Rₐ, R_{b}, R_{c} and Rₑ is of the same group, the number of carbon atoms in each of Rₐ, R_{b}, R_{c} and Rₑ may be the same or different.
R_{c} may be a C₁₋₃₀alkyl, a C₁₋₁₂alkyl, a C₁₋₁₀alkyl, or a C₁₋₆alkyl. R_{c} may be a methyl, ethyl, propyl, butyl, pentyl, hexyl, heptyl, octyl, nonyl decyl, undecyl or dodecyl.
R_{d} may be an optionally substituted ester having the formula —R_{g}′CO₂R_{g}, an optionally substituted amide having the formula —R_{g}′CONHR_{g} or an optionally substituted alkoxy. R_{g}′ and R_{g} may each be independently selected from the group consisting of an C₁₋₃₀alkylene, C₂₋₃₀alkenylene, C₂₋₃₀alkynylene, C₁₋₃₀-heteroalkylene, C₂₋₃₀-heteroalkenylene and C₂₋₃₀-heteroalkynylene. R_{g} may be C₂-alkylene and R_{g}′ may be C₃-alkenylene. Where R_{d} is an optionally substituted alkoxy, R_{d} may be ethoxycarbonyl.

The polymer may be a copolymer that further comprises another monomer having a hydrophilic moiety, a vinyl monomer or combinations thereof. This monomer may comprise one or more hydrophilic moiety selected from the group consisting of hydroxyl, carboxyl, carbonyl, acryloyl, amines and imines. This monomer may be selected from the group consisting of N-isopropylacrylamide, acrylamide, 2-oxaline, ethyleneimine, acrylic acid, methacrylate, ethylene glycol, ethylene oxide, vinyl alcohol, vinylpyrrolidinone. The vinyl monomer may have the general formula (11) CH₂═CRₓR_{y}, wherein Rₓ and R_{y} are each independently selected from the group consisting of hydrogen, optionally substituted aliphatic, an alicyclic, an aromatic, an arylaliphatic, an heterocycloalkyl and a heteroaryl.

The polymer may further comprise repeating monomer units of formula (2):

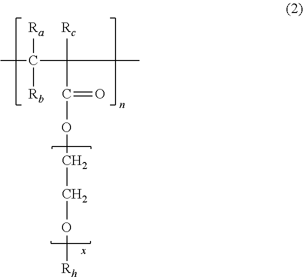

wherein Rₐ, R_{b}, R_{c}, are as defined above;
Rₕ is selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl;
n is at least 1; and
x is a number selected from 1 to 50.

The polymer may be selected from the group consisting of Formula (3), Formula (4), Formula (5), Formula (6), Formula (7), Formula (8), Formula (9) and Formula (10):

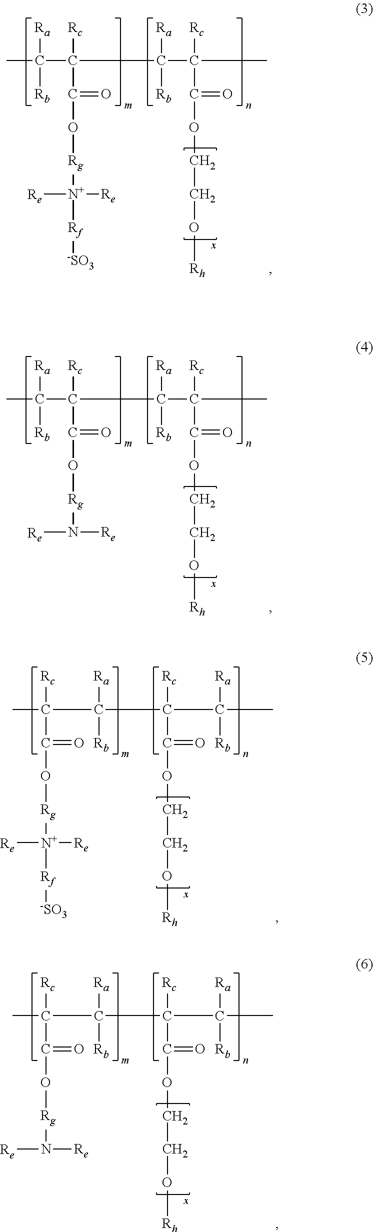

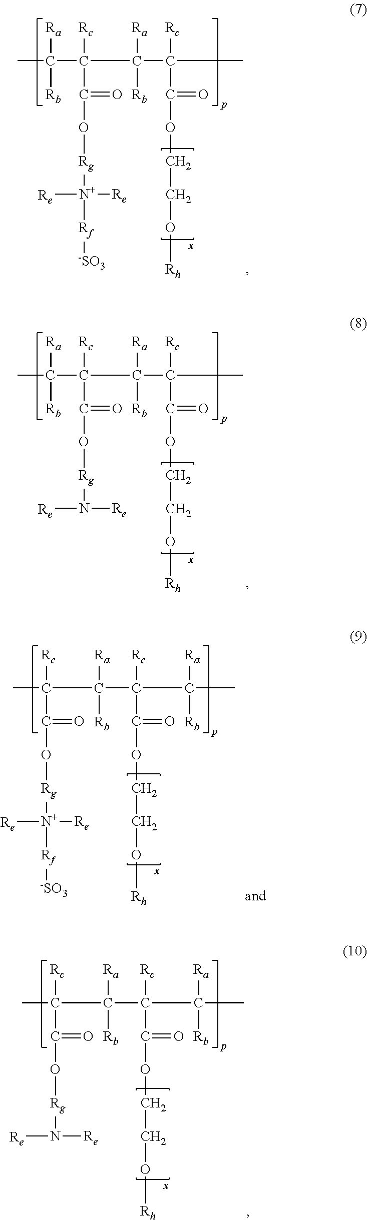

wherein Rₐ, R_{b}, R_{c}, R_{d}, Rₑ, R_{f}, Rₕ, R_{g}, m, n and x are as above; and
p is at least 1.

The polymer may be of Formula (9) and may be poly(dimethyl(methaacryloyloxyethyl)ammonium propanesulfonate)-co-poly(polyethylene glycol methacrylate).

The polymer may be of Formula (10) and may be poly(dimethylamino ethyl methacrylate)-co-poly(polyethylene glycol methacrylate).

The polymer may have a molecular weight selected from the range of about 5 kDa to about 500 kDa, about 5 kDa to about 50 kDa, about 5 kDa to about 100 kDa, about 5 kDa to about 200 kDa, about 5 kDa to about 300 kDa, about 5 kDa to about 400 kDa, about 50 kDa to about 500 kDa, about 100 kDa to about 500 kDa, about 200 kDa to about 500 kDa, about 300 kDa to about 500 kDa, or about 400 kDa to about 500 kDa.

The polymer may be coupled to the shell via a bridging group selected from the group consisting of an aminoalkyl silane, haloalkyl silane, mercapto alkyl silane and an aminoalkoxysilane.

The particle may contain about 10% to about 90% (w/w), about 10% to about 30% (w/w), about 10% to about 50% (w/w), about 10% to about 70% (w/w), about 30% to about 90% (w/w), about 50% to about 90% (w/w), about 70% to about 90% (w/w), or about 20% to about 80% (w/w), of the polymer of formula (1).

The particle may be a microparticle or a nanoparticle. The particle may have a hydrodynamic diameter in the range of about 1 nm to about 100 μm, about 1 nm to about 500 nm, about 1 nm to about 1 μm, about 1 nm to about 10 μm, about 1 nm to about 50 μm, about 500 nm to about 100 μm, about 1 μm to about 100 μm, about 10 μm to about 100 μm, about 50 μm to about 100 μm, or about 10 nm to about 10 μm.

The pores of the particle may be microporous or mesoporous. The pores on the particle may have an average diameter between about 1 nm to about 50 nm, about 1 nm to about 5 nm, about 1 nm to about 10 nm, about 1 nm to about 20 nm, about 1 nm to about 30 nm, about 1 nm to about 40 nm, about 5 nm to about 50 nm, about 10 nm to about 50 nm, about 20 nm to about 50 nm, about 30 nm to about 50 nm, or about 40 nm to about 50 nm.

The core of the particle may define an inner void.

The pores of the particle may alternate between an “open” position and a “closed” position in response to an external stimulus. The external stimulus may be selected from temperature or carbon dioxide concentration. Hence, changes in the external temperature and/or carbon dioxide concentration may cause a physical change in the chain length of the polymer such that the polymer may adopt an uncoiled (or extended) configuration or a collapsed configuration.

As a result of the change in the polymer chain length, the contents of the core (which can contain an agent, a load or a cargo) can be exchanged or prevented from being exchanged with the external environment. As such, when in the “open” position, the core-shell particle may allow the exchange of core contents agent between the core of the particle and the external environment. When in the “closed” position, the core-shell particle may not allow the exchange of the agent between the core of the particle and the external environment.

Where the external stimulus is a change in the temperature, at a temperature above a critical temperature, the pores are in the “open” position and when the temperature is below the critical temperature, the pores are in said “closed” position. The rate of release of the agent from the core-shell particle may be increased with an increase in the temperature. The critical temperature may be in the range of about 35° C. to about 45° C., about 35° C. to about 37° C., about 35° C. to about 39° C., about 35° C. to about 41° C., about 35° C. to about 43° C., about 37° C. to about 45° C., about 39° C. to about 45° C., about 41° C. to about 45° C., about 43° C. to about 45° C., or about 37° C. to about 38° C. The polymer that is capable of responding to the change in the temperature may be poly(dimethyl(methaacryloyloxyethyl)ammonium propanesulfonate)-co-poly(polyethylene glycol methacrylate), which has a critical temperature of about 37.5° C.

Where the external stimulus is a change in the carbon dioxide concentration, in the presence of carbon dioxide, the pores may be in the “open” position and when carbon dioxide is substantially absent, the pores are in the “closed” position. The polymer that is responsive to carbon dioxide may be poly(dimethylamino ethyl methacrylate)-co-poly(polyethylene glycol methacrylate).

The contents of the core may be an agent. The agent may be a fluid or may be an active ingredient dispersed in a fluid.

The particle may comprise of silica. Where the core of the particle is a void, the core-shell particle may be a hollow particle in which the shell comprises silica.

The core-shell particle may be made by polymerizing the monomer of Formula (1) onto the shell of a hollow particle. The hollow particle may be a hollow silica particle. The silica surface may be functionalized with a halide. The halide functionality then serves as an atomic transfer radical polymerization (ATRP) initiating moiety. This is done by a sequential surface modification of hydroxyl groups to primary amine groups by reacting with an organo-silane coupling agent. The organo-silane coupling agent may have amine functional groups and alkoxy functional groups. The primary amine groups may then be functionalized with halide groups by reacting with an ATRP initiator.

The organo-silane coupling agent may be selected from the group consisting of epoxysilane, mercaptosilane, alkylsilane, phenylsilane, ureidosilane and vinylsilane, titanium based compounds, aluminum chelates, and aluminum/zirconium based compounds. Exemplary organo-silane coupling agents include silane coupling agents such as β-(3,4-epoxycyclohexyl) ethyltrimethoxysilane, γ-glycidoxypropyltrimethoxysilane, 3-glycidoxypropylmethyldimethoxysilane, γ-mercaptopropyltrimethoxysilane, aminopropyltrimethoxysilane, γ-aminopropylmethyldimethoxysilane, γ-aminopropyltriethoxysilane, γ-aminopropylmethyldiethoxysilane, γ-(N, N-dimethyl) aminopropyltrimethoxysilane, γ-(N, N-diethyl)aminopropyltrimethoxysilane, γ-(N,N-dibutyl) aminopropyltrimethoxysilane, γ-(N-methyl)anilinopropyltrimethoxysilane, γ-(N-ethyl) anilinopropyltrimethoxysilane, γ-(N, N-dimethyl) aminopropyltriethoxysilane, γ-(N, N-diethyl) aminopropyltriethoxysilane, γ-(N,N-dibutyl)aminopropyltriethoxysilane, γ-(N-methyl)aminopropyltriethoxysilane, γ-(N-ethyl)aminopropyltriethoxysilane, γ-(N, N-dimethyl) aminopropylmethyldimethoxysilane, γ-(N,N-diethyl)aminopropylmethyldimethoxysilane, γ-(N,N-dibutyl)aminopropylmethyldimethoxysilane, γ-(N-methyl)aminopropylmethyldimethoxysilane, γ-(N-ethyl)amiopropylmethyldimethoxysilane, N-(trimethoxysilylpropyl) ethylenediamine, N-(dimethoxymethylsilylisopropyl) ethylenediamine, or γ-mercaptopropylmethyldimethoxysilane. These may be used alone or in combination of two or more thereof.

Exemplary ATRP initiator may include 2-bromoisobutyryl bromide, 2-azidoethyl 2-bromoisobutyrate, bis[2-(2′-bromoisobutyryloxy)ethyl]disulphide, bis[2-(2-bromoisobutyryloxy)undecyl]disulphide, 2-bromoisobutanoic acid N-hydroxysuccinimide ester, 2-bromoisobutyric anhydride, 2-(2-bromoisobutyryloxy)ether methacrylate, tert-butyl 2-bromoisobutyrate, 3-butynyl 2-bromoisobutyrate, dipentaerythritol hexakis(2-bromoisohutyrate), dodecyl 2-bromoisobutyrate, ethyl 2-bromoisobutyrate, ethylene bis(2-bromoisobutyrate), 2-hydroxyethyl 2-bromoisobutyrate, 1-(DL-1,2-isopropylideneglyceryl) 2-bromoisobutyrate, methyl 2-bromoisobutyrate, octadecyl 2-bromoisobutyrate, pentaerythritol tetrakis(2-bromoisobutyrate), 1-(phthalimidomethyl) 2-bromoisobutyrate, poly(ethylene glycol) bis(2-bromoisobutyrate), poly(ethylene glycol) methyl ether 2-bromoisobutyrate, propargyl 2-bromoisobutyrate, 1,1,1-tris(2-bromoisobutyryloxymethyl)ethane or 10-undecenyl 2-bromoisobutyrate. Hence, the halide groups on the hollow particle may be bromide groups such that the halide-functionalized particle is a bromide-functionalized particle.

Once the hollow silica particles are modified with the halide groups, surface initiated ATRP may be performed. Here, the halide modified particles may be polymerized with a precursor monomer that results in the formation of the repeating monomer units of Formula (1) in the presence of an organic solvent and a copper catalyst (such as copper bromide). The precursor monomer to the repeating monomer units of Formula (1) may be a vinyl conjugated —R_{d}—X(Rₑ)—Rᵣ—Y (wherein R_{d}, X, Rₑ, R_{f} and Y are as defined herein for Formula (1)). An additional precursor monomer may be added to the polymerization reaction, such as one that results in the formation of the repeating monomer units of Formula (2). The precursor monomer to the repeating monomer units of Formula (2) may be a vinyl conjugated —C(═O)O—[CH₂—CH₂—O]ₓ—Rₕ (where x and Rₕ are as defined herein for Formula (2)).

Other methods of synthesizing or polymerizing the core-shell particle may be used. Such methods may be conventional polymerization methods. The core-shell particle may be formed from thiol-lactam initiated radical polymerization, in which the surface of the hollow particle is functionalized with a mercaptosilane and polymerized with the precursor monomer(s) as mentioned above in the presence of a lactam (such as for example butyrolactam).

There is also provided a method of transferring an agent into or out of the core of the core-shell particle as defined herein. The method may comprise the step of altering the temperature or carbon dioxide concentration of a solution that is in contact with the core-shell particle. Changes in the temperature and/or carbon dioxide of the solution then results in the opening or closing of the pores on the shell of the core-shell particle, thus allowing for the entry or egress of the agent into or from the core of the core-shell particle. The agent may be a water-soluble or insoluble ingredient.

There is also provided a method of loading a gas into the core of the core-shell particle as defined herein. The method may comprise the step of exposing the core-shell particle to a gaseous environment containing the gas and allowing the gas to permeate into the core of the core-shell particle. The gas may be oxygen gas. The gas may then be stored within the core of the core-shell particle which can then be released from the core when the particle is placed in an environment where carbon dioxide is prevalent. For example, the gas filled core-shell particle may be injected into a patient's bloodstream and can deliver the gas (which is oxygen gas) intravenously, especially when the core-shell particles are exposed to dissolved carbon dioxide. This may serve as a nano-sized breathing apparatus that is capable of providing oxygen to a patient, such as to the tissues, organs, or blood vessels of a patient. For example, the nano-sized breathing apparatus may be capable of oxygenating a patient's blood while providing medical workers a larger timeframe in which to take action to restore a patient's breathing.

There is also provided a delivery agent comprising a plurality of core-shell particles as defined herein, wherein the core of the particles contains an agent. The agent may be a chemical compound, molecule or a gas.

There is also provided a pharmaceutical composition comprising a plurality of core-shell particles as defined herein, wherein the core of the particles contains an agent. The agent may be a therapeutic agent or may be oxygen. The therapeutic agent may be a drug, protein, or gene.

The core-shell particle can be pre-synthesized or pre-loaded with the agent, thus affording a longer shelf life.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1(a) is a transmission electron microscopy (TEM) image showing an unmodified hollow silica sphere (HSS) of Example 1. The scale bar in FIG. 1(a) is 0.5 μm.

FIG. 1(b) is a TEM image of HSS-graft-(poly(DMAEMA)-co-poly(PEGMA_{1.1k})) particle of Example 1. The scale bar in FIG. 1(b) is 100 nm.

FIG. 2 is a graph showing the thermal gravimetric analyses of pristine HSS, HSS-Br and HSS-graft-(PDMAEMA-co-PPEGMA_{1.1k}) of Example 1.

FIG. 3(a) is a TEM image of pristine hollow silica nanoparticle of Example 2. The scale bar in FIG. 2(a) is 100 nm.

FIG. 3(b) is a TEM image of HSi-graft-(poly(DMAEMA)-co-poly(PEGMA_{1.1k})) particle of Example 2. The scale bar in FIG. 2(b) is 100 nm.

FIG. 4 is a graph showing the thermal gravimetric analyses of pristine HSi, HSi—Br, HSi-graft-(PDMAEMA-co-PPEGMA_{1.1k}) of Example 2 and that of HSi-graft-(PDMAPS-co-PPEGMA_{1.1k}) of Example 3.

FIG. 5(a) is an optical microscope image of oxygen filled HSS-graft-(PDMAEMA-co-PPEGMA_{1.1k}) of Example 4 in PBS at a concentration of 1 mg/mL. FIG. 5(a) was taken at 100× magnification.

FIG. 5(b) is an optical microscope image of oxygen filled HSS-graft-(PDMAEMA-co-PPEGMA_{1.1k}) of Example 4 in PBS at a concentration of 1 mg/mL that has been bubbled with carbon dioxide for 60 minutes. FIG. 5(b) was taken at 100× magnification.

FIG. 6(a) is a graph showing the measured dissolved oxygen content against time for a degassed PBS solution when an equal volume of O₂ saturated PBS (▪) or O₂ loaded nanoparticle solution () was added at t=3 minutes as indicated by the blue arrow as demonstrated in Example 4. Solid lines indicate fitted exponential decay function from t=7 minutes.

FIG. 6(b) is a schematic diagram showing the change in the structure of the nanoparticle in the presence of carbon dioxide.

FIG. 7 is a graph showing the relative cell viability in L929 cell line after exposure to HSS-graft-polymer (nanoparticles) and positive control polymers, PEI and PDMAEMA (0.03125-1 mg/mL). Relative cell viability was determined using the MTT Assay and expressed as a percentage of untreated cells (control) as demonstrated in Example 5.

FIG. 8(a) is a graph showing the normalized fluorescence measurements of collected filtrate showing the drug release profiles of Rhodamine B loaded nanoparticles as demonstrated in Example 6.

FIG. 8(b) is a schematic diagram showing the change in the structure of the nanoparticle in response to a change in temperature.

FIG. 9 is a graph showing the dynamic light scattering (DLS) results showing the change in the hydrodynamic radius of the particles in PBS and deionized water with respect to temperature according to Example 6.

### EXAMPLES

Non-limiting examples of the invention and a comparative example will be further described in greater detail by reference to specific Examples, which should not be construed as in any way limiting the scope of the invention.

All precursors/chemicals used in the Examples were obtained from Sigma-Aldrich of St. Louis of Missouri of the United States of America.

### Example 1—Synthesis of HSS-Graft-(Poly(DMAEMA)-Co-Poly(PEGMA_{1.1k}))

### Synthesis of Hollow Silica Spheres (HSS)

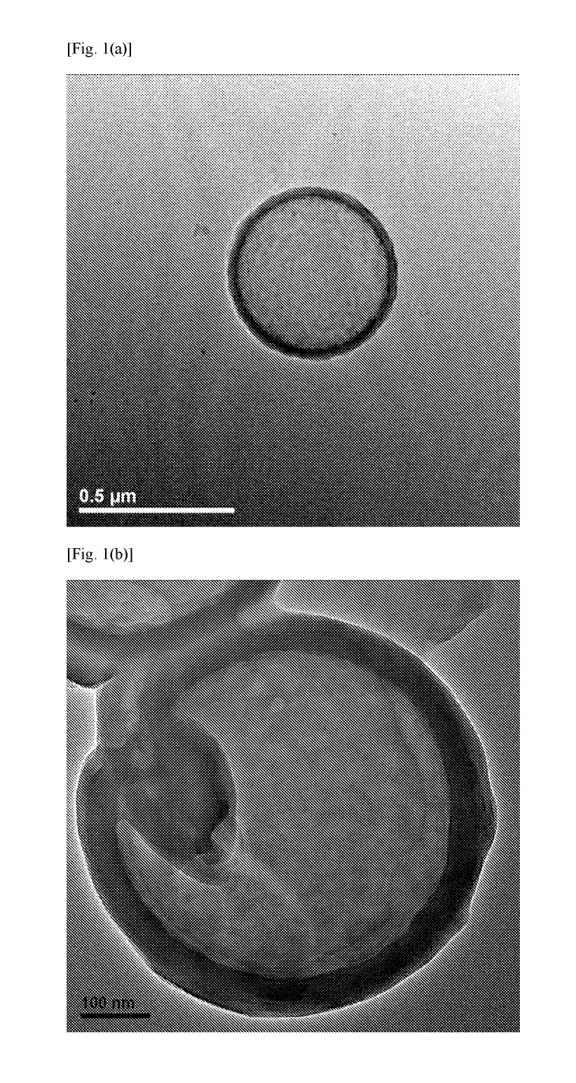

0.5 μm sized hollow silica spheres (HSS) were synthesized by firstly dissolving 0.5 g of polyvinyl pyrrolidone (Mₙ=40,000 g·mol⁻¹) in deionized water (100 mL) and stirring at room temperature for 4 hours. To this, 11 g of styrene (which had been pre-treated with basic alumina to remove inhibitor) and 0.26 g of 2,2′-azobis(2-methylpropionamide) dihydrochloride was added and degassed with argon for 30 minutes, then allowed to react for 24 hours at 70° C. The mixture was cooled at the end of the reaction, and 18 mL of the mixture was extracted and mixed with 240 mL of ethanol and 12 mL of aqueous ammonia (25 wt %). Separately, 3.2 mL of tetraethylorthosilicate was mixed with 5 mL of ethanol and added dropwise to the mixture of polystyrene colloid, ethanol and ammonia at 50° C. This mixture was then allowed to react for 24 hours. The solution was centrifuged to collect the suspended particles, washed three times with ethanol, then dried, followed by calcination in a furnace at 550° C. The particles were visualized by transmission electron microscopy (TEM) and as shown in FIG. 1(a), the particle size of the HSS was approximately 500 nm, with a 50 nm silica shell. The particles were also characterized by BET which showed that HSS had a surface area of 230 m²/g and an average pore size of 45 Å.

### Bromide Functionalization of HSS (HSS-Br) for Atom-Transfer Radical Polymerization

0.2 g of the HSS obtained above were dispersed in 10 mL of p-xylene, followed by the addition of 0.3 mL of 3-amino propyl triethoxy silane. The mixture was stirred for 24 hours at 90° C. under argon, after which the mixture was washed several times with diethyl ether, and then filtered. The collected solids were dried in a vacuum oven, then redispersed in 40 mL of anhydrous chloroform and 1.2 mL of triethylamine in a large round bottom flask. This flask was immersed in ice while 0.6 mL of 2-bromoisobutyryl bromide in 4 mL anhydrous chloroform was added dropwise over a period of 1 hour, after which the reaction mixture was removed from ice and allowed to react at ambient temperature for a further 18 hours. At the end of the reaction, the solids were filtered and washed several times with chloroform, then dried under vacuum.

### Synthesis of HSS-Graft-(Poly(DMAEMA)-Co-Poly(PEGMA_{1.1k}))

A monomer solution was first prepared by adding 4 g (25.5 mmol) of dimethyl amino ethyl methacrylate (DMAEMA) and 0.4 g (0.36 mmol) of poly(ethylene glycol) methacrylate (Mₙ=1,100, PEGMA_{1.1k}) (both of which had been pre-treated with basic alumina to remove inhibitor) to 5 mL of anhydrous anisole in a sealed flask. This solution was purged with nitrogen gas for 1 hour. The bromide functional HSS (0.2 g) were first dispersed and sonicated in 5 mL of anhydrous anisole in a schlenk flask. To this, 0.02 g of copper bromide was added. Before use, copper bromide was purified by refluxing in glacial acetic acid for 18 hours, washed with ether and dried extensively under vacuum. The flask was then sealed and purged with nitrogen gas for 30 minutes. After 30 minutes, 40 μL of N,N,N′,N′,N″-Pentamethyl diethylenetriamine (PMDETA) (which was distilled before use) was added to the schlenk flask via a gas tight syringe, and the solution was purged with nitrogen for a further 30 minutes. At the end of this time period, the monomer solution was transferred to the schlenk fast via a cannula, and the flask was reacted at 90° C. for 9 hours. The overall reaction scheme for this polymerization is shown in Scheme 1 below.

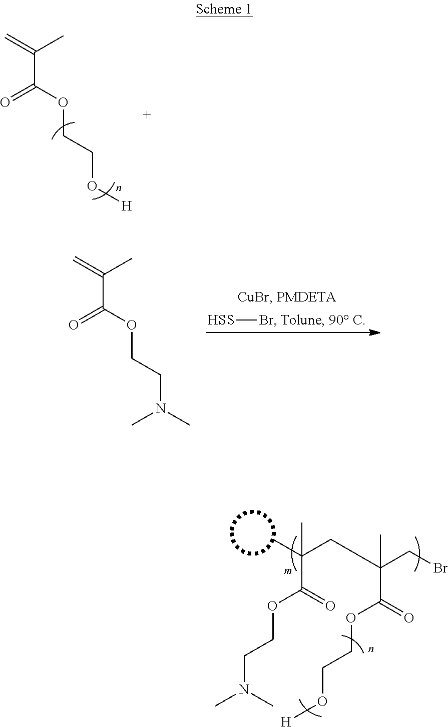

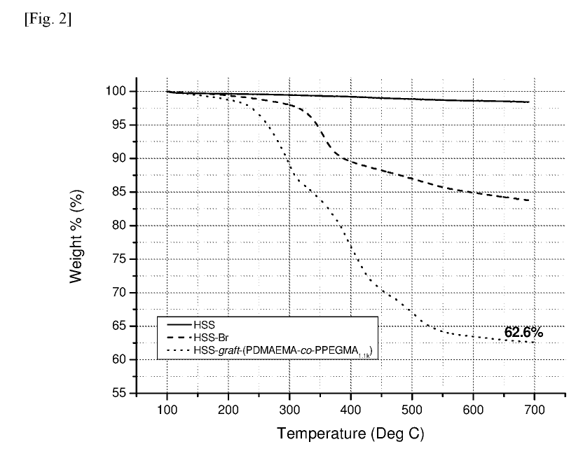

The product was then centrifuged and washed with acetone to remove the solvent, copper and unreacted monomer. To further remove the copper catalyst, the solids were redispersed in acetone and mixed with Dowex Marathon MSC (H⁺) ion exchange resin. The washed solids were dried under vacuum to yield the nanoparticles. The HSS-graft-(PDMAEMA-co-PPEGMA_{1.1k}) particles were visualized by TEM and shown in FIG. 1(b). As shown in FIG. 1(b), the diameter of the particle ranges from 585 nm to 615 nm and the shell thickness of the particle ranges from 10 to 75 nm. The pristine HSS, HSS-Br and HSS-graft-(PDMAEMA-co-PPEGMA_{1.1k}) particles were analyzed for the weight percent of polymer by thermogravimetric analysis (TGA) as shown in FIG. 2. The TGA results show that approximately 37.4% of the polymer grafted hollow silica was made up of the initiator and the polymer.

### Example 2—Synthesis of HSi-Graft-(Poly(DMAEMA)-Co-Poly(PEGMA_{1.1k}))

### Synthesis of Hollow Silica (HSi)

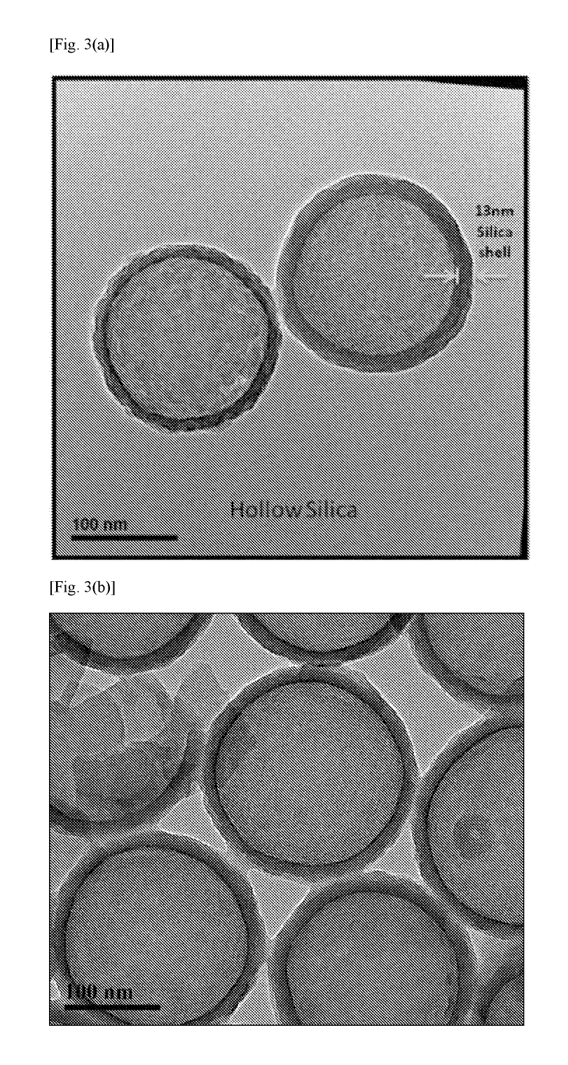

Hollow silica (HSi) with a diameter of 150 nm was synthesized and functionalized via the procedure described by Lay et al. In a typical process, 3.0 g of polyvinyl pyrrolidone was dissolved in 100 mL of HPLC grade water under stirring for 24 hours at room temperature. Then, 11.0 mL of styrene and 0.26 g of 2,2′-azobis(2-methylpropionamide) dihydrochloride were added to the solution under stirring at 100 rpm and 70° C. under argon. After 24 hours, 18 mL of polystyrene colloid solution was mixed with 240 mL of ethanol and an 12 mL of aqueous solution of ammonia (25 wt %). Then, 3.18 mL of tetraethyl orthosilicate in 5 mL of ethanol was added dropwise, and the mixture was stirred at 50° C. for 24 hours. The solid was collected by centrifugation and was calcinated at 550° C. to get hollow silica spheres. The particles were visualized by TEM and as shown in FIG. 3(a), the particle size of the HSi was approximately 150 nm, with a 13 nm silica shell. The particles were also characterized by BET which showed that HSi had a surface area of 401 m²/g and an average pore size of 55 Å.

### Synthesis of HSi-Graft-(Poly(DMAEMA)-Co-Poly(PEGMA

[S&F: Please advise on the process used to form the HSi-graft-polymer as your TD only focuses on HSS as the template. If the process to form the HSi-graft-polymer is the same as that to form the HSS-graft-polymer, we can state it as such here. If this is the case, please check that the amounts of the chemicals used, reaction parameters (temperature, time, etc) are the same as those for the HSS process.

The HSi-graft-(poly(DMAEMA)-co-poly(PEGMA_{1.1k})) particles were visualized by TEM and shown in FIG. 3(b). As shown in FIG. 3(b), the particles have an approximate diameter of about 231 nm and an approximate shell thickness of about 18 nm.

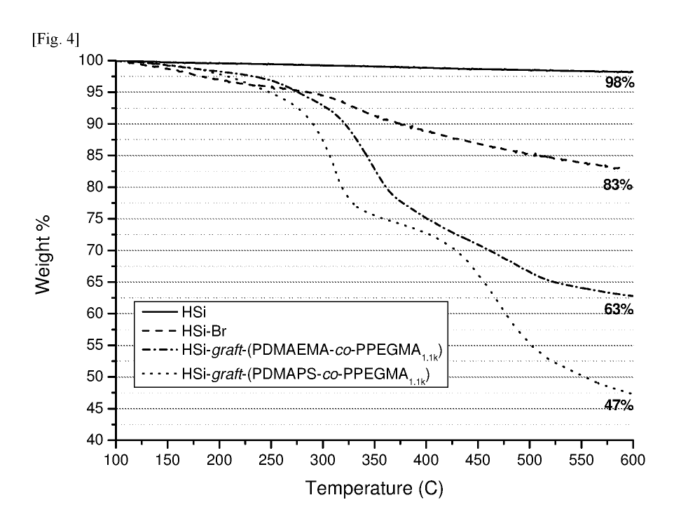

The pristine HSi, HSi—Br and HSi-graft-(PDMAEMA-co-PPEGMA_{1.1k}) particles were analyzed for the weight percent of polymer by TGA as shown in FIG. 4. The TGA results show that the polymer content by weight of the HSi-graft-(PDMAEMA-co-PPEGMA_{1.1k}) was 37%.

### Example 3—Synthesis of HSi-Graft-(P(DMAPS)-Co-P(PEGMA_{1.1k}))

To a 100 mL roundbottom flask, 50 mg of HSi-graft-(P(DMAEMA)-co-P(PEGMA_{1.1k})) was dispersed in 50 mL of tetrahydrofuran (THF). To this, 50 mg (36 μL) of 1,3-propane sultone was added and the mixture was reacted at 60° C. for 24 hours. The pendant tertiary amine moieties on the polymer then under betainization. The overall reaction scheme for this polymerization if shown in Scheme 2.

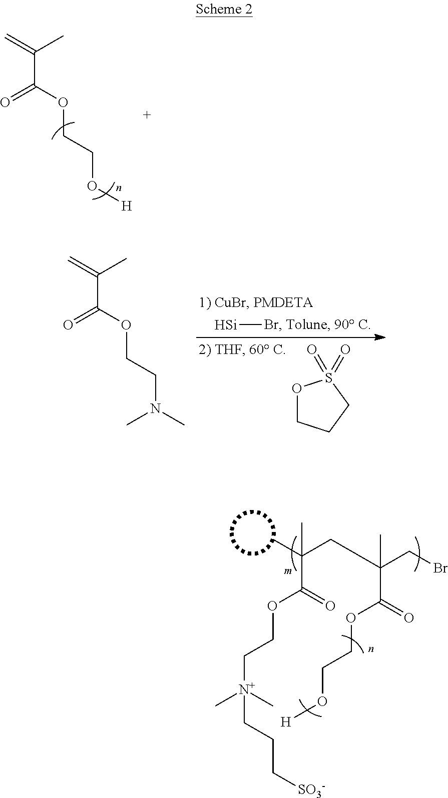

At the end of the reaction time, the product was centrifuged, and subjected to 3 cycles of redispersion in THF and centrifugation before the collected pellet was redispersed in water. The aqueous solution was freeze dried to yield the zwitterionic HSi-graft-(P(dimethyl(methaacryloyloxyethyl)ammonium propanesulfonate)-co-P(PEGMA_{1.1k})). The HSi-graft-(PDMAPS-co-PPEGMA_{1.1k}) was then subjected to TGA and the result is shown together with the other HSi particles in FIG. 4. The final polymer content on the HSi-graft-(PDMAPS-co-PPEGMA_{1.1k}) particles was 53% which showed that the increase in the polymer content of the particles could be attributed to the complete betanization of the tertiary amines to the zwitterionic moiety.

### Example 4—Responsiveness of HSS-Graft-(Poly(DMAEMA)-Co-Poly(PEGMA_{1.1k})) Particles

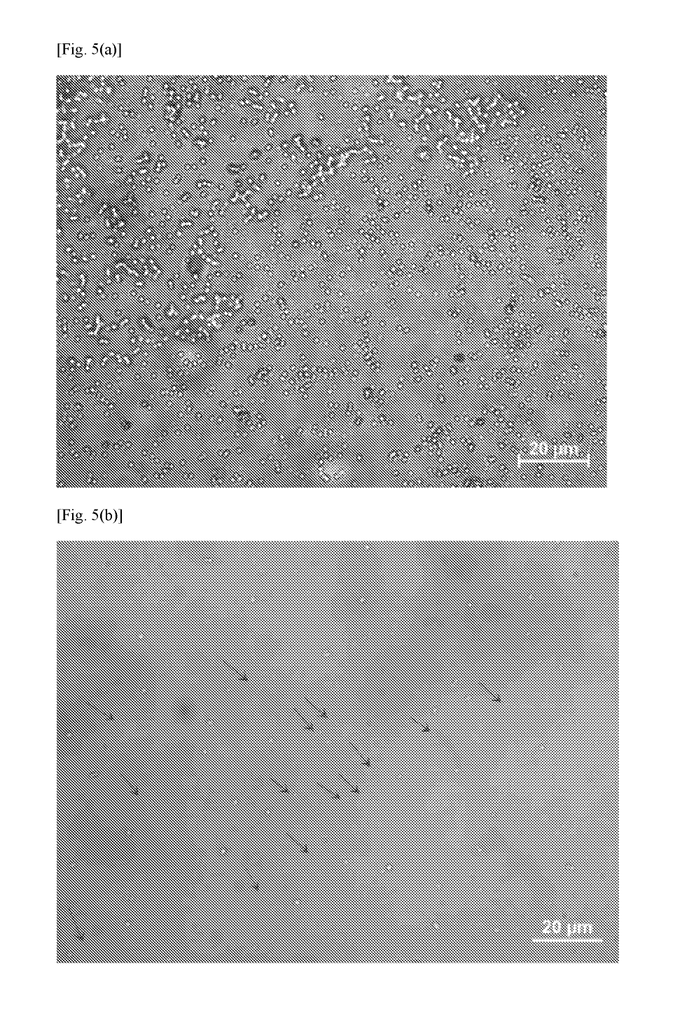

The HSS-graft-(poly(DMAEMA)-co-poly(PEGMA_{1.1k})) nanoparticles were placed in a sealed schlenk flask and placed under vacuum for 3 hours to ensure full evacuation of air from the nanoparticles. After the three hours, the schlenk flask was backfilled with oxygen gas at 1 atmosphere and left to equilibrate for 2 hours. Separately, PBS was sparged with nitrogen gas for 1 hour to remove dissolved gases. The degassed PBS was then transferred to the schlenk flask to make up a 10 mg/mL solution of oxygen filled nanoparticles. These particles were further diluted to 1 mg/mL for optical microscopy, the image of which is shown in FIG. 5(a). In FIG. 5(a), multiple bubble-like features can be observed, which are the oxygen filled particles and are present in this manner due to the difference in refractive index of the particle due to the transition of light from liquid to gas and back to liquid. The 1 mg/mL solution was then bubbled with carbon dioxide for 60 minutes, after which a sample was viewed under the optical microscope again, the image of which is shown in FIG. 5(b). In FIG. 5(b), significantly fewer bubble-like features were visible, and the ones visible appeared to be less bright, as highlighted by the arrows in FIG. 5(b). The reduction of these features suggest that the liquid-filled polymer-HSS particles were not visible by optical microscope due to low contrast and only the presence of a gas allowed for their visualization by optical microscopy.

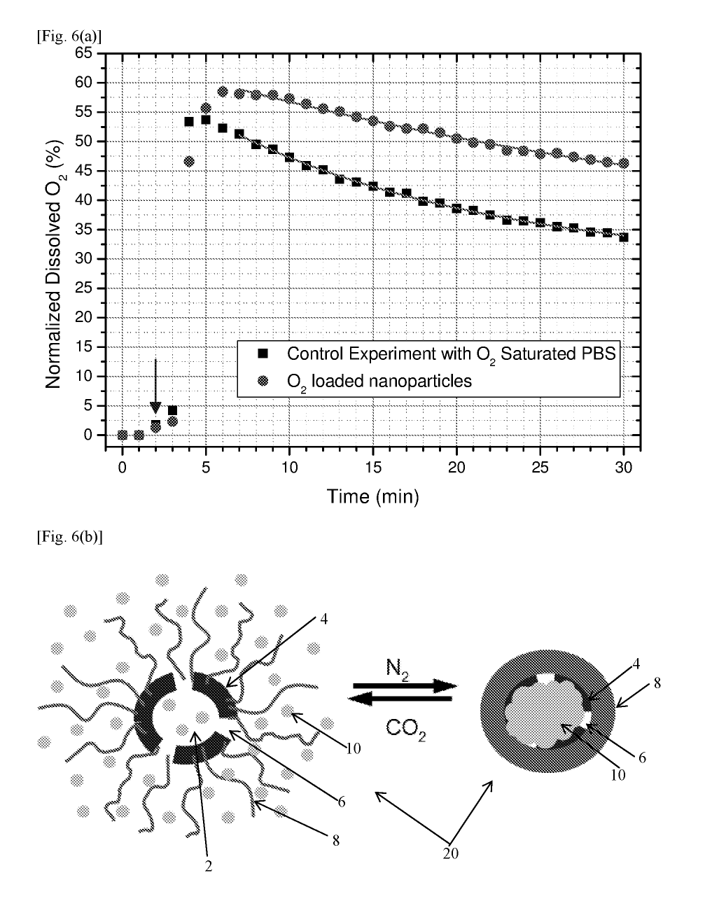

Dissolved oxygen studies were performed on a Rank Brothers Digital Model 10 dissolved oxygen meter that was calibrated with oxygen saturated PBS solution. Nitrogen sparged solution of PBS was first placed in the oxygen sensor to measure the baseline oxygen content of degassed PBS (which was 19.5%). The solution was allowed to equilibrate for 3 minutes after which an equal volume of the oxygen-filled nanoparticle solution was added to the PBS. The dissolved oxygen content was measured every minute over 30 minutes. As a control, the experiment was repeated with oxygen saturated PBS instead of the nanoparticle solution. The normalized results are shown in FIG. 6a.

When the O₂ loaded nanoparticle solution was added, the dissolved oxygen levels of the solution continued to increase for 3 minutes. It plateaued for an additional 3 minutes after which the oxygen levels began to decrease slowly over time. Conversely, when the O₂ saturated PBS solution was added, the dissolved oxygen level rose immediately, was stable for 1 minute, and then began a steady decline. While there was no specific dosage of carbon dioxide to the solutions, carbon dioxide would still be present due to the exposure of the sample cell to the atmosphere.

The oxygen saturation curve for the control experiment suggested that the oxygen levels equilibrated with the atmosphere and thus decline. Furthermore, it is known that the oxygen sensor also consumed dissolved oxygen, thereby contributing to the decline of dissolved O₂. This decay can be regarded as the baseline decay of dissolved oxygen in solution. For the control system, a first order exponential decay was fitted to the decay portion of the curve from t=6 minutes, and is mathematically described by Math. 1. In the case of the O₂ loaded nanoparticles, the maximum O₂ concentration was only achieved 3 minutes after the addition of the solution. This thereby suggested that O₂ was continually being released from the nanoparticles. A first order exponential decay is fitted to the decay curve from t=6 minutes and is mathematically described by Math. 2.

$$\begin{matrix}y = 34.6 {e}^{( - \frac{x}{16.2} )} + 28.6 ( {R}^{2} = 0.9978 ) & [ Math . 1 ] \\ y = 34.7 {e}^{( - \frac{x}{39} )} + 29.8 ( {R}^{2} = 0.9916 ) & [ Math . 2 ]\end{matrix}$$

It is noted that the two equations show that the decay of dissolved oxygen over time as slower for the O₂ loaded nanoparticles (Equation 2) as compared to the control experiment with the addition of saturated O₂ PBS (Equation 1). The exponential decay constant was almost 2.5 times higher in the control compared to the decay in the presence of O₂ loaded nanoparticles. This result suggested that O₂ was still continually released from the nanoparticles up to 30 minutes and even beyond.

The mechanism of the response of the nanoparticles to carbon dioxide is shown in FIG. 6(b). In FIG. 6(b), the nanoparticle 20 is a core-shell particle which has a core 2 surrounded by a silica shell 4 with pores 6 extending from the core 2 through the silica shell 4. The polymer brushes 8 are bonded to the outer surface of the silica shell 4. In the presence of carbon dioxide, the polymer brushes 8 grafted to the surface of the silica shell 4 (namely the PDMAEMA) respond to the carbon dioxide by uncoiling and exposed the silica pores 6. This allows any oxygen 10 present in the core 2 of the nanoparticle 20 to be released from the nanoparticle 20. In the absence of carbon dioxide, the grafted polymers 8 collapse to form a layer that ‘seal’ the silica pores 6, thus encapsulating the oxygen 10 within the core 2 of the nanoparticle 20.

The particle size in water was then characterized by dynamic light scattering (DLS). For the HSS-graft-polymer particles, 1 mg/mL solution of the nanoparticles in degassed (nitrogen) deionized water was prepared. Particle size measurements were taken over 10 readings and were found to be 1287±49 nm. The solution was then bubbled with carbon dioxide for 2 minutes and the particle size was re-measured. The new particle size after carbonation was found to be 1548±43 nm. The hydrodynamic particle size increased by approximately 260 nm thereby suggesting that the carbonation had an effect on the polymer brush. This showed that PDMAEMA ionized and increased in solubility in the presence of carbon dioxide.

### Example 5—Cytotoxicity of HSS-Graft-(Poly(DMAEMA)-Co-Poly(PEGMA_{1.1k})) Particles

Cytotoxicity of the nanoparticles was assessed on L929 mouse fibroblast cells (obtained from American Type Culture Collection of Rockville of Maryland of the United States of America). Cells were maintained in Dulbecco's Modified Eagles Medium (DMEM) supplemented with 10% heat-inactivated fetal bovine serum (FBS), 100 units/mg penicillin, 100 μg/mL streptomycin at standard conditions, 37° C., 5% CO₂ and 95% relative humidity. The cells were seeded in 96-well cell culture plates (1×10⁵ cells/mL) and incubated for 24 hours.

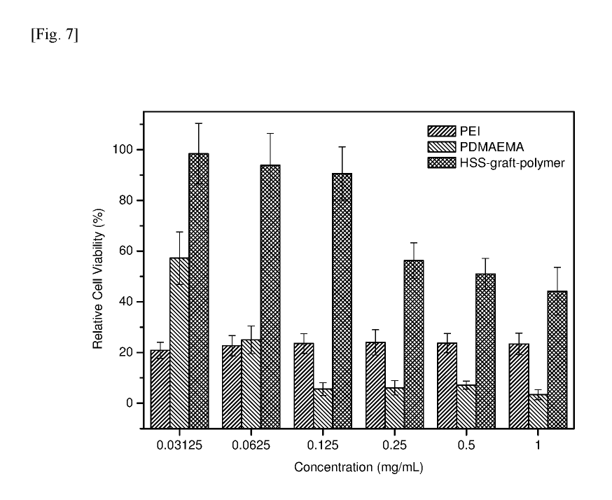

The cell culture medium was then replaced with medium containing serial dilutions of the nanoparticle or control polymers, Poly(dimethylaminoethyl methacrylate) (PDMAEMA) and polyethylenimine (PEI), (0.03125-1 mg/mL) and incubated for 4 hours. PDMAEMA and PEI are positive control polymers that are commonly used in gene transfection studies and were used here for comparative purposes. The culture medium was then removed and replaced with fresh DMEM and incubated for 42 hours. 10 μL of filtered 1-(4,5-dimethyl-thiazol-2-yl)-3,5-diphenylfor-mazan (MTT, obtained from Sigma-Aldrich of St. Louis of Missouri of the United States of America) (5 mg/mL) in PBS (pH 7.4) was added and cells were incubated for an additional 4 hours. Unreacted dye was removed by aspiration and formazan crystals were dissolved with 100 μL/well DMSO and the absorbance was measured at 570 nm (SpectraMax Plus384, Molecular Devices). 6 replicate measurements were performed and the relative cell viability is expressed as: [A_{570 (polymer+cells)}−A_{570 (blank)}]/[A_{570 (cells only)}−A_{570 (blank)}]×100% and shown in FIG. 7.

As shown in FIG. 7, for the nanoparticle solution, 90-100% of viable cells were detected at the lower concentration range tested (0.03125-0.125 mg/mL) while the relative cell viability ranges between 45-55% at higher concentrations (0.25-1 mg/mL). This was significantly less toxic than PDMAEMA where the cell viability rapidly declined from 55% (at 0.03125 mg/mL) to below 10% and for PEI where even at exposure to the lowest concentration resulted in less than 25% cell viability. The particles were fairly benign at low concentrations thereby showing the vast potential of this material as an oxygen delivery vehicle. Carbon dioxide responsive polymers are an emerging class of materials where its clinical potential has not been fully realized and therefore the toxicity of these materials could be lowered further via polymer modifications.

### Example 6—Thermoresponsiveness of HSi-Graft-(P(DMAPS)-Co-P(PEGMA_{1.1k})) Particles

10 mg of HSi-(P(DMAPS)-co-P(PEGMA_{1.1k})) nanoparticles was weighed into 5 mL of deionized water and sonicated for 10 minutes to fully disperse all the particles. 10 mg of rhodamine B was then introduced into the solution and the mixture was stirred for 18 hours at 50° C. After this period, the solution was cooled to 5° C. The solution was centrifuged and the supernatant was discarded. The remnant solids were washed with cold deionized water several times to remove excess rhodamine B, and the solids were eventually freeze dried to yield a pink powder.

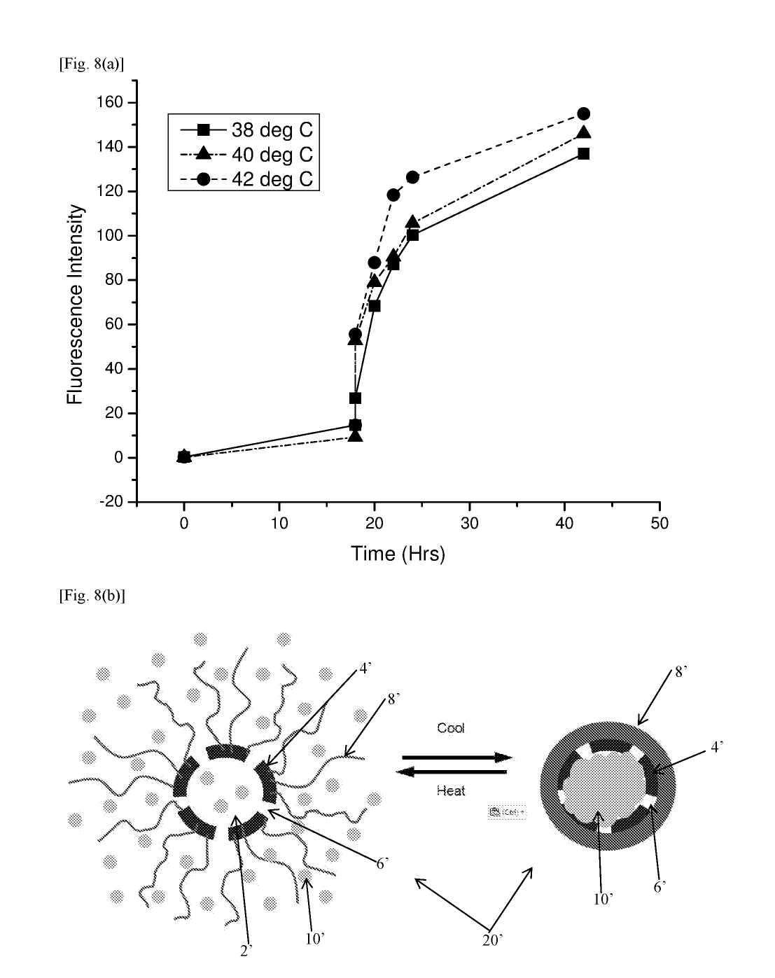

The lypholized nanoparticles were then dissolved into three batches of water at ambient temperature and stirred for 18 hours. A sample was taken initially and at 18 hours, filtered to remove the particles, and then tested for its fluorescence. After 18 hours, the solution batches were heated up to (i) 38° C.; (ii) 40° C.; and (iii) 42° C.; in order to obtain the drug release profiles at 3 different temperatures. The samples were taken immediately, and at 2 hour intervals 3 times, followed by a final sample at 24 hours after heating. Similarly, the samples were filtered immediately and analyzed for the fluorescence of released Rhodamine B, with an excitation wavelength of 552 nm, and an emission wavelength of 572 nm. The normalized fluorescence measurements of the collected filtrate showing the drug release profiles of Rhodamine B loaded nanoparticles are reported in FIG. 8(a). As seen from FIG. 8(a), the release profile of rhodamine B from the nanoparticles shows that the rhodamine B was released from the particles when the temperature of the particles was elevated beyond a critical temperature (as will be discussed further below). This shows the thermoresponsive properties of the particles.

The mechanism of the response of the nanoparticles to changes in temperature is shown in FIG. 8(b). The elements in FIG. 8(b) are similar to the elements in FIG. 6(b) and hence, like reference numerals will be used to denote like elements but with an additional prime (′). In FIG. 8(b), the nanoparticle 20′ is a core-shell particle which has a core 2′ surrounded by a silica shell 4′ with pores 6′ extending from the core 2′ through the silica shell 4′. The polymer brushes 8′ are bonded to the outer surface of the silica shell 4′. At a temperature above a critical temperature, the polymer brushes 8′ grafted to the surface of the silica shell 4′ (namely the PDMAPS) respond to the elevated temperature by uncoiling and exposing the silica pores 6′. This allows any agent/load 10′ present in the core 2′ of the nanoparticle 20′ to be released from the nanoparticle. At a temperature below the critical temperature, the grafted polymers 8′ collapse to form a layer that ‘seal’ the silica pores 6′, thus encapsulating the agent/load 10′ within the core 2′ of the nanoparticle 20′.

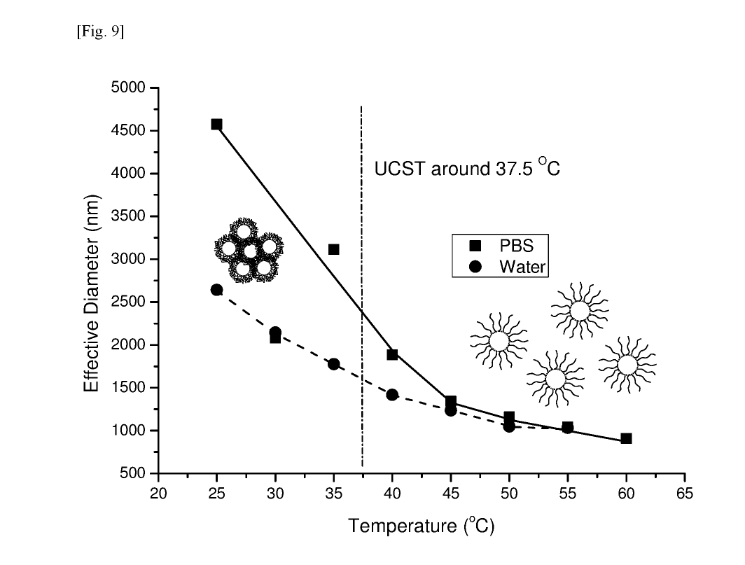

To determine the critical temperature of the nanoparticle, particularly, the upper critical solubility temperature (UCST), DLS measurements were conducted at a range of temperatures for the zwitterionic polymer grafted HSi in both deionized water and PBS at a concentration of 1 mg/mL since ionic strength can potentially affect the thermoresponsive characteristics. The DLS measurements are shown in FIG. 9. Here, the change in the hydrodynamic radius of the particles in PBS and deionized water with respect to temperature can be observed. As a median point of size change can be seen at 37.5° C. irrespective of solvent, it was determined that the UCST is approximately 37.5° C.

In traditional stimuli responsive polymeric systems, the solubility is often regarded as when the chains are fully extended and solubilized, and insolubility is regarded as when the chains are collapsed. Therefore, in terms of the hydrodynamic radius of nanoparticles, the particle size would increase with an increase in solubility. Therefore, based on the DLS results shown in FIG. 9, could suggest that there was a decrease in polymer solubility with increasing temperature. However, it is likely that the insolubility of the polymeric chains at a lower temperature caused the aggregation of particles, which resulted in the higher particle size measurement, and as the polymer chains uncoiled, the particles dispersed and thus there was a decrease in the recorded particle size. This is further illustrated by the scheme within FIG. 9 which shows aggregated hollow silica particles with a collapsed polymer layer on the left side of the graph, and dispersed hollow silica with fully extended polymeric brushes on the right side of the graph.

Hence, it has been shown that the HSi-graft-(P(DMAPS)-co-P(PEGMA_{1.1k})) particles are thermoresponsive.

### INDUSTRIAL APPLICABILITY

The core-shell particle may be used as a delivery agent for delivering an agent, a cargo or a load into an environment which triggers the release of the agent, cargo, or load from within the core-shell particle to the external environment.

The core-shell particle may be used as a delivery agent for a therapeutic agent. The core-shell particle may be injected or ingested by a patient and upon reaching the critical temperature (which can be the patient's normal body temperature or higher in the case of a fever), the core-shell particle can release the therapeutic agent in vivo. The core-shell particle can also be used as a delivery agent in skincare products. The core-shell particle can be formulated as a nanocapsule for sustained deliver of the agent to the skin of a patient during topical administration. The core-shell particle may be used as detergent additives.

Where the agent is a gas such as oxygen gas, the core-shell particle may be injected into a patient and serves as a nano-sized breathing apparatus by releasing oxygen from the core-shell particle when the particle is exposed to dissolved carbon dioxide. This then results in the oxygenation of blood in the patient and provides medical workers with more time to stabilise the patient. This may be useful at times of cardiac thoracic arrest to give medical workers a longer window to seek medical intervention, thereby reducing mortality rates. The core-shell particle can be used in sports medicine to provide direct oxygen administration; in defense to allow naval divers to function without scuba gear; or in stroke victims to oxygenate the blood vessels in the brain.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A core-shell particle having pores extending through its shell and a plurality of polymers that are bonded to the outer surface of the shell, wherein each polymer is a copolymer comprising repeating monomer units of formulae (1) and (2): 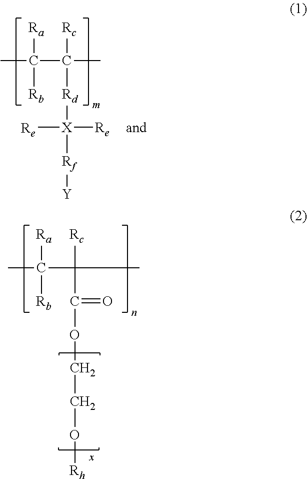
wherein
m and n are at least 1;
Rₐ, R_{b}, R_{c} and Rₑ are independently selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl;
R_{d} is selected from the group consisting of a bond, optionally substituted hetero-aliphatic, optionally substituted aliphatic group, optionally substituted alkoxy, carbonyl, optionally substituted ester, optionally substituted amide, optionally substituted aryl, optionally substituted heteroaryl, optionally substituted cycloalkyl and optionally substituted heterocycloalkyl;
X is selected from the group consisting of a heteroatom or a positively charged ion;
R_{f} and Y are absent when X is a heteroatom but are both present when X is a positively charged ion such that R_{f} is selected from the group consisting of optionally substituted aliphatic, optionally substituted hetero-aliphatic, optionally substituted aryl, optionally substituted heteroaryl, optionally substituted cycloalkyl and optionally substituted heterocycloalkyl; and Y is a negatively charged ion when bonded to R_{f}; and
Rₕ is selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl.

2. The core-shell particle according to claim 1, wherein said optionally substituted aliphatic is selected from the group consisting of optionally substituted C₁₋₃₀alkylene, optionally substituted C₂₋₃₀alkenylene and optionally substituted C₂₋₃₀alkynylene.

3. The core-shell particle according to claim 1, wherein said optionally substituted hetero-aliphatic contains one or more heteroatoms selected from N, O, S, Se or Si, and is selected from the group consisting of optionally substituted C₁₋₃₀-heteroalkylene, optionally substituted C₂₋₃₀-heteroalkenylene and optionally substituted C₂₋₃₀-heteroalkynylene.

4. The core-shell particle according to claim 1, wherein X is selected from the group consisting of N, N⁺ and P⁺ or when X is N⁺ or P⁺, Rₜ and Y are present and Y is selected from the group consisting of sulfonate, carboxylate, nitrite and carbonite.

5. (canceled)

6. The core-shell particle according to claim 4, wherein R_{f} is selected from the group consisting of C₂-alkylene, C₃-alkylene, C₄-alkylene, C₅-alkylene and C₆-alkylene.

7. The core-shell particle according to claim 1, wherein said Rₐ, R_{b}, R_{c} and Rₑ are independently a hydrogen or an optionally substituted aliphatic.

8. (canceled)

9. (canceled)

10. The core-shell particle according to claim 1, wherein said R_{d} is an optionally substituted ester having the formula —R_{g}′CO₂R_{g} or an optionally substituted amide having the formula —R_{g}′CONHR_{g} wherein R_{g}′ and R_{g} is each independently selected from the group consisting of an C₁₋₃₀alkylene, C₂₋₃₀alkenylene, C₂₋₃₀alkynylene, C₁₋₃₀heteroalkylene, C₂₋₃₀-heteroalkenylene and C₂₋₃₀-heteroalkynylene.

11. (canceled)

12. The core-shell particle according to claim 1, wherein said polymers are independently selected from the group consisting of Formula (3), Formula (4), Formula (5), Formula (6), Formula (7), Formula (8), Formula (9) and Formula (10): 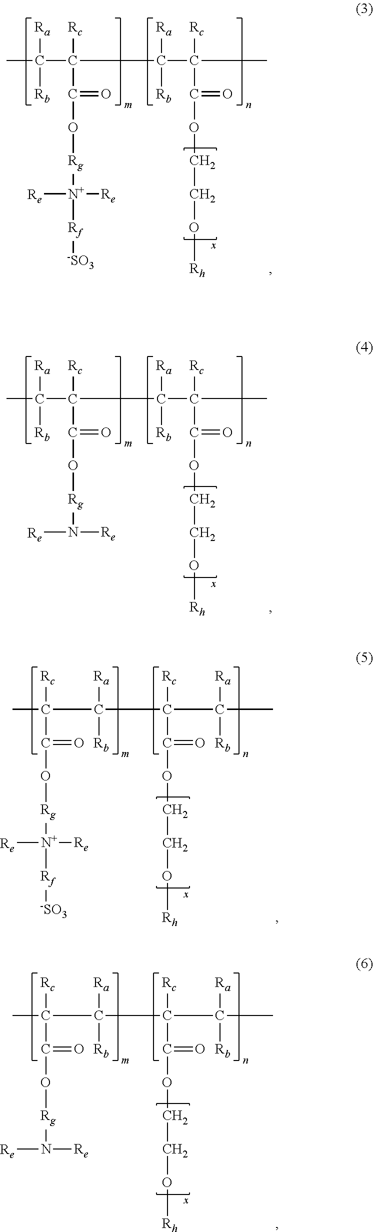 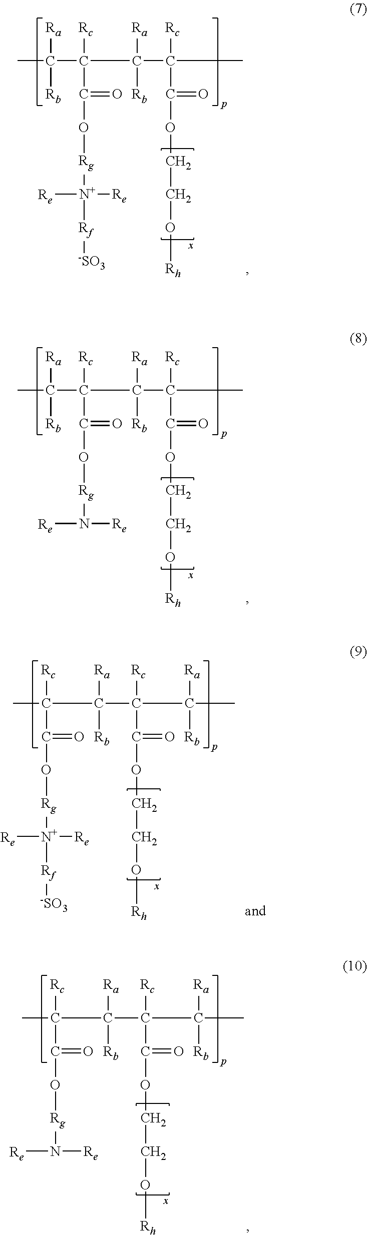
wherein m and n are at least 1;
Rₐ, R_{b}, R_{c} and Rₑ are independently selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl;
R_{d} is selected from the group consisting of a bond, optionally substituted hetero-aliphatic, optionally substituted aliphatic group, optionally substituted alkoxy, carbonyl, optionally substituted ester, optionally substituted amide, optionally substituted aryl, optionally substituted heteroaryl, optionally substituted cycloalkyl and optionally substituted heterocycloalkyl;
X is selected from the group consisting of a heteroatom or a positively charged ion;
R_{f} and Y are absent when X is a heteroatom but are both present when X is a positively charged ion such that R_{f} is selected from the group consisting of optionally substituted aliphatic, optionally substituted hetero-aliphatic, optionally substituted aryl, optionally substituted heteroaryl, optionally substituted cycloalkyl and optionally substituted heterocycloalkyl; and Y is a negatively charged ion when bonded to R_{f}; and
Rₕ is selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl; and
p is at least 1.

13. The core-shell particle of claim 12, wherein said polymer of formula (9) is poly(dimethyl(methacryloyloxyethyl)ammonium propanesulfonate)-co-poly(polyethylene glycol methacrylate).

14. The core-shell particle of claim 12, wherein said polymer of formula (10) is poly(dimethylamino ethyl methacrylate)-co-poly(polyethylene glycol methacrylate).

15. The core-shell particle according to claim 1, wherein said polymer has a molecular weight selected from the range of 5 kDa to 500 kDa.

16. The core-shell particle according to claim 1, wherein said polymer is coupled to the shell via a bridging group selected from the group consisting of an aminoalkyl silane, haloalkyl silane, mercapto alkyl silane and an aminoalkoxysilane.

17. The core-shell particle according to claim 1, wherein said particle contains 10% to 90% (w/w) of the polymer of formulae (1) and (2), optionally wherein said article is a microparticle or a nanoparticle with a hydrodynamic diameter of 1 nm to 100 μm.

18. (canceled)

19. The core-shell particle according to claim 1, wherein the pores of said particle are microporous or mesoporous.

20. The core-shell particle according to claim 1, wherein said pores on said particle alternate between an “open” position and a “closed” position in response to an external stimulus, or wherein said external stimulus is selected from temperature or carbon dioxide concentration.

21. (canceled)

22. The core-shell particle according to claim 20, wherein when said temperature is above a critical temperature, said pores are in said “open” position and when said temperature is below said critical temperature, said pores are in said “closed” position, or wherein when said polymer is poly)dimethyl(methaacryloyloxyethyl)ammonium propanesulfonate)-co-poly(polyethylene glycol methacrylate), said critical temperature is 37.5° C.

23. (canceled)

24. (canceled)

25. The core-shell particle according to claim 20, wherein when said carbon dioxide is present, said pores are in said “open” position and when said carbon dioxide is substantially absent, said pores are in said “closed” position, or wherein when said polymer is poly(dimethylamino ethyl methacrylate)-co-poly(polyethylene glycol methacrylate), said polymer is responsive to carbon dioxide.

26. (canceled)

27. (canceled)

28. A method of synthesizing a core-shell particle having pores extending through its shell and a plurality of polymers that are bonded to the outer surface of the shell, wherein each polymer is a copolymer comprising repeating monomer units of formulae (1) and (2): 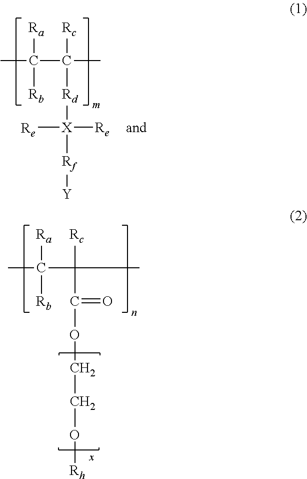
wherein
m and n are at least 1;
Rₐ, R_{b}, R_{c} and R_{g} are independently selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl;
R_{d} is selected from the group consisting of a bond, optionally substituted hetero-aliphatic, optionally substituted aliphatic group, optionally substituted alkoxy, carbonyl, optionally substituted ester, optionally substituted amide, optionally substituted aryl, optionally substituted heteroaryl, optionally substituted cycloalkyl and optionally substituted heterocycloalkyl;
X is selected from the group consisting of a heteroatom or a positively charged ion;
R_{f} and Y are absent when X is a heteroatom but are both present when X is a positively charged ion such that R_{f} is selected from the group consisting of optionally substituted aliphatic, optionally substituted hetero-aliphatic, optionally substituted aryl, optionally substituted heteroaryl, optionally substituted cycloalkyl and optionally substituted heterocycloalkyl; and Y is a negatively charged ion when bonded to R_{f}; and
Rₕ is selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl, comprising:
i) providing a halide-functionalized particle; and
ii) reacting said halide-functionalized particle with a monomer of formulae (1) and (2) to form said core-shell particle.

29. The method of claim 28, wherein said halide-functionalized particle is a bromide-functionalized particle.

30-32. (canceled)

33. A pharmaceutical composition comprising a plurality of core-shell particles having pores extending through its shell and a plurality of polymers that are bonded to the outer surface of the shell, wherein each polymer is a copolymer comprising repeating monomer units of formulae (1) and (2): 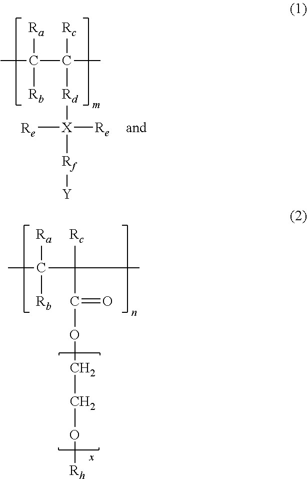
wherein
m and n are at least 1;
Rₐ, R_{b}, R_{c} and R_{g} are independently selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl;
R_{d} is selected from the group consisting of a bond, optionally substituted hetero-aliphatic, optionally substituted aliphatic group, optionally substituted alkoxy, carbonyl, optionally substituted ester, optionally substituted amide, optionally substituted aryl, optionally substituted heteroaryl, optionally substituted cycloalkyl and optionally substituted heterocycloalkyl;
X is selected from the group consisting of a heteroatom or a positively charged ion;
R_{f} and Y are absent when X is a heteroatom but are both present when X is a positively charged ion such that R_{f} is selected from the group consisting of optionally substituted aliphatic, optionally substituted hetero-aliphatic, optionally substituted aryl, optionally substituted heteroaryl, optionally substituted cycloalkyl and optionally substituted heterocycloalkyl; and Y is a negatively charged ion when bonded to R_{f}; and
Rₕ is selected from the group consisting of a hydrogen, optionally substituted aliphatic, optionally substituted hetero-aliphatic, optionally substituted aryl, optionally substituted heteroaryl, optionally substituted cycloalkyl and optionally substituted heterocycloalkyl, wherein the core of said particles contains an agent.
